(19) United States
(12) Patent Application Publication
(10) Pub. No.: US 2017/0269071 A1
(43) Pub. Date: Sep. 21, 2017

(54) LIQUID-SAMPLE COMPONENT ANALYSIS METHOD

(71) Applicant: OLYMPUS CORPORATION, Tokyo (JP)

(72) Inventor: Akira SATO, Tokyo (JP)

(73) Assignee: OLYMPUS CORPORATION, Tokyo (JP)

(21) Appl. No.: 15/611,265

(22) Filed: Jun. 1, 2017

Related U.S. Application Data
(63) Continuation of application No. PCT/JP2014/082163, filed on Dec. 4, 2014.

(51) Int. Cl. G01N 33/543

(52) U.S. Cl. CPC G01N 33/543

(57) ABSTRACT

A liquid-sample component analysis method includes a dripping step of dripping a liquid sample onto a flat horizontal surface, a drying step of drying a liquid droplet of the liquid sample formed on the horizontal surface while keeping the liquid droplet still so as to obtain a plurality of concentrically-arranged ring-shaped deposits formed on the horizontal surface and composed of components having different particle diameters, and a measuring step of measuring a vibrational spectrum in each region including only one of the deposits so as to individually acquire vibrational spectra of the plurality of deposits.

### CROSS-REFERENCE TO RELATED APPLICATIONS

This is a continuation of International Application PCT/JP2014/082163, with an international filing date of Dec. 4, 2014, which is hereby incorporated by reference herein in its entirety.

### TECHNICAL FIELD

The present invention relates to liquid-sample component analysis methods.

### BACKGROUND ART

In the related art, biochemical methods and optical methods are used for analyzing particulate components, such as cells, bacteria, proteins, and viruses, contained in liquid samples, such as body fluids extracted from biological organisms or cell or tissue culture solutions.

Examples of the biochemical methods used include the ELISA method in which color or light is detected based on an antigen-antibody reaction of a protein, the PCR method in which the messenger RNA is amplified and determined, and a two-dimensional electrophoresis method in which the DNA is cleaved and fragmented (for example, see Patent Literature 1). These biochemical methods are advantageous in that the analytical precision is high.

Known optical methods include drop coating deposition spectroscopy, surface-enhanced Raman scattering (SERS), and surface-enhanced infrared absorption (SEIRA) (for example, see Non Patent Literature 1 and Patent Literatures 2 and 3). In an optical method in which the vibrational spectrum of a sample is measured, as in Non Patent Literature 1 and Patent Literatures 2 and 3, rapid analysis is normally possible, but the analytical sensitivity is lower than that in a biochemical method. Hence, the liquid sample is mixed with metallic nano-particles, such as gold, silver, or copper, and the vibrational spectrum from the particulate components in the liquid sample is enhanced in accordance with a plasmon resonance effect between the metallic nano-particles and probe light. Moreover, in drop coating deposition spectroscopy, the liquid sample is dripped onto a substrate and dried thereon so that the particulate components in the liquid sample are concentrated, thereby enhancing the detection sensitivity.

### CITATION LIST

### Patent Literature

### {PTL 1}

Japanese Translation of PCT International Application, Publication No. 2006-515420

### {PTL 2}

U.S. Pat. No. 7,889,334

### {PTL 3}

Japanese Translation of PCT International Application, Publication No. Hei 11-507724

### Non Patent Literature

### {NPL 1}

FILIK, Jacob, et al., “Drop coating deposition Raman spectroscopy of protein mixtures”, Analyst, 23 Apr. 2007, 132, p. 544-550

### SUMMARY OF INVENTION

An aspect of the present invention provides a liquid-sample component analysis method including a dripping step of dripping a liquid sample onto a flat horizontal surface; a drying step of drying a liquid droplet of the liquid sample formed on the horizontal surface as a result of the dripping step while keeping the liquid droplet still so as to obtain a plurality of concentrically-arranged ring-shaped deposits formed on the horizontal surface and composed of components having different particle diameters; and a measuring step of measuring a vibrational spectrum in each region including only one of the deposits so as to individually acquire vibrational spectra of the plurality of deposits.

### BRIEF DESCRIPTION OF DRAWINGS

FIG. 1 illustrates the overall configuration of a liquid analyzing apparatus used in a liquid-sample component analysis method according to an embodiment of the present invention.

FIG. 2 is a perspective view illustrating a substrate used in the liquid-sample component analysis method according to the embodiment of the present invention.

FIG. 3 is a flowchart illustrating the liquid-sample component analysis method according to the embodiment of the present invention.

FIG. 4 is a side view illustrating a drying process of a liquid sample in a drying step in FIG. 3.

FIG. 5 schematically illustrates movement of particulate components in the drying process in FIG. 3.

FIG. 6 is a plan view of the substrate showing a dried mark of a liquid droplet formed on a flat surface as a result of the drying step.

FIG. 7 illustrates an intensity profile obtained as a result of a preliminary measuring step.

FIG. 8 is a plan view of the substrate showing measurement regions set in a measurement-region setting step.

FIG. 9 is a side view illustrating the structure of a flat surface of a substrate used in a first modification of the liquid-sample component analysis method according to the embodiment of the present invention.

FIG. 10 is a flowchart illustrating the first modification of the liquid-sample component analysis method according to the embodiment of the present invention.

FIG. 11 illustrates a state where a particular component has fallen onto the flat surface in FIG. 9.

FIG. 12 is a flowchart illustrating a second modification of the liquid-sample component analysis method according to the embodiment of the present invention.

FIG. 13 illustrates three types of antibody-modified micro-particles used in an antibody-modified-particle mixing step in FIG. 12.

FIG. 14 is a plan view of deposit rings formed by the three types of micro-particles in FIG. 13.

FIG. 15 illustrates three types of metal-modified micro-particles used in the antibody-modified-particle mixing step in FIG. 12.

FIG. 16 is a plan view of deposit rings formed by the three types of micro-particles in FIG. 13 and the three types of micro-particles in FIG. 15.

### DESCRIPTION OF EMBODIMENTS

A liquid-sample component analysis method according to an embodiment of the present invention will be described below with reference to the drawings.

First, a liquid analyzing apparatus 1 used for performing the liquid component analysis method according to this embodiment will be described.

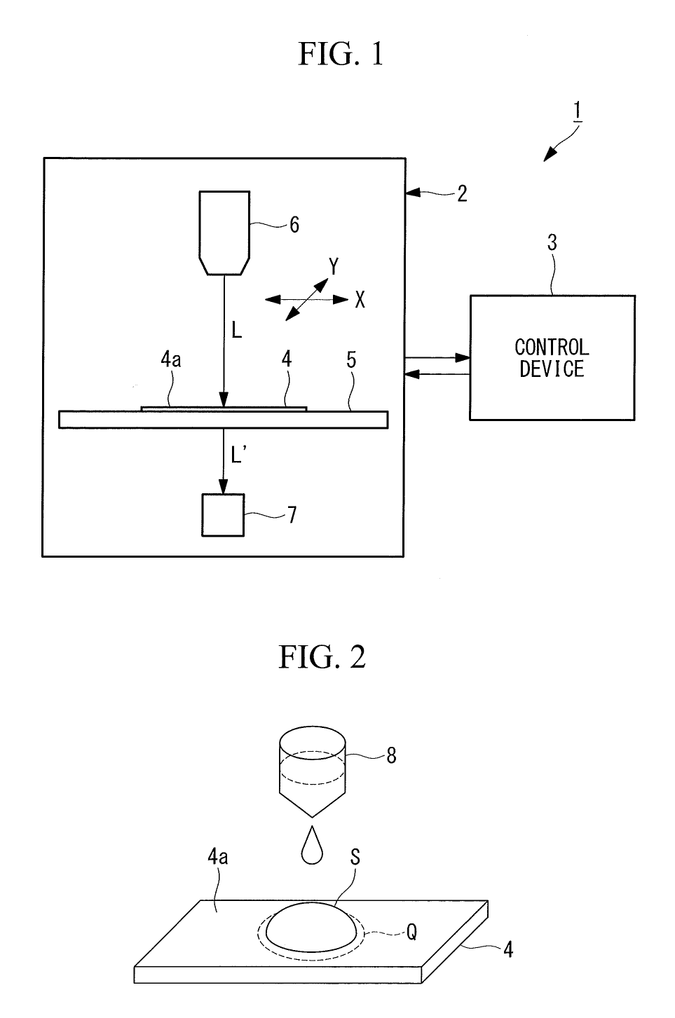

As shown in FIG. 1, the liquid analyzing apparatus 1 includes a microscope 2 that acquires a vibrational spectrum of a sample and a control device 3 that analyzes the vibrational spectrum acquired by the microscope 2 and that also controls the microscope 2 based on the analytical result.

As shown in FIG. 2, a substrate 4 has a smooth flat surface 4a. A predetermined dripping range Q indicating a position where a liquid sample S should drip is set on the flat surface 4a. The flat surface 4a has high hydrophilicity and high wettability at least in the dripping range Q, so that the liquid sample S dripping onto the dripping range Q spreads evenly on the flat surface 4a. Preferred examples of the substrate 4 having high wettability include a quartz-glass substrate and a substrate whose surface is coated with metal, such as gold, silver, or aluminum. In order to enhance wettability, the flat surface 4a may be chemically modified so as to be coated with a hydrophilic group, such as a hydroxyl group or an amino group, or may have small nanometer-size protrusions and recesses formed thereon to increase the surface area.

The liquid sample S is a body fluid extracted from a biological organism, such as lacrima, saliva, or perspiration, or a cell or tissue culture solution. The liquid sample S contains a plurality of types of particulate components having different particle diameters, such as cells, bacteria, mycoplasmas, viruses, and proteins (biopolymers).

The microscope 2 includes a stage 5 having a load surface on which the substrate 4 is placed, a focusing lens 6 disposed above the stage 5 so as to face the load surface, and a detector 7 that detects signal light L′ from the particulate components contained in the sample. Laser light in a visible or infrared wavelength range from a laser light source (not shown) is output as probe light L, and the probe light L is focused onto the sample on the substrate 4 by the focusing lens 6. The signal light L′ emitted from the sample as a result of it being irradiated with the probe light L is detected and split by the detector 7. The signal light L′ is, for example, Raman-scattered light or infrared light partially absorbed by the sample. Consequently, a vibrational spectrum, such as a Raman scattering spectrum or an infrared absorption spectrum, is acquired. The focusing lens 6 and the stage 5 are provided in such a manner as to be movable relative to each other in the horizontal direction, so that the irradiation position of the probe light L from the focusing lens 6 to the sample can be moved in the horizontal direction.

The control device 3 is, for example, a computer equipped with a CPU (central processing unit) and a storage device. The storage device stores therein a program for executing predetermined processing on data of the vibrational spectrum acquired by the microscope 2 and for determining and executing a subsequent operation of the microscope 2 based on the processing result.

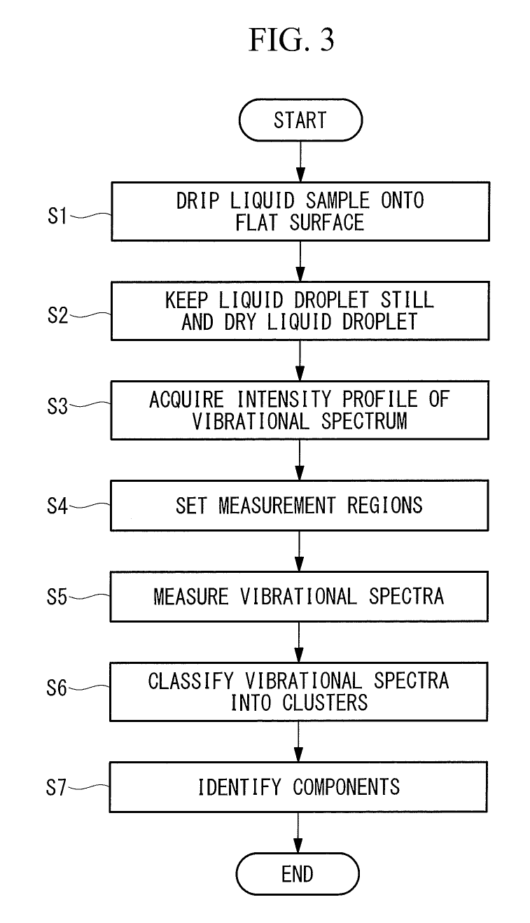

As shown in FIG. 3, the liquid-sample component analysis method according to this embodiment includes a dripping step S1 of dripping the liquid sample S onto the flat surface 4a, a drying step S2 of drying a liquid droplet of the liquid sample S formed on the flat surface 4a so as to form particulate-component deposits R within the dripping range Q, a preliminary measuring step S3 of measuring the spatial distribution of the deposits R within the dripping range Q, a measurement-region setting step S4 of setting measurement regions based on the measurement result obtained in the preliminary measuring step S3, a measuring step S5 of measuring vibrational spectra in the measurement regions, a classifying step S6 of classifying the acquired vibrational spectra in accordance with multivariate analysis, and an identifying step S7 of identifying the particulate components from the vibrational spectra.

First, in the dripping step S1, a small amount of the liquid sample S is dripped onto the dripping range Q of the horizontally-disposed flat surface (horizontal surface) 4a by using a dripping device 8, such as a pipette, thereby forming a liquid droplet within the dripping range Q. In this case, the liquid sample S dripped on the flat surface 4a forms the liquid droplet by spreading evenly on the flat surface 4a until a contact angle θ with the flat surface 4a reaches a predetermined angle.

The contact angle θ is defined by the Young equation (1):

γSV=γLV*cos θ+γSL (1)

where γSV denotes surface tension between the flat surface 4a and the air, γLV denotes surface tension between the liquid sample S and the air, and γSL denotes surface tension between the flat surface 4a and the liquid sample S. In this embodiment, it is preferable that the contact angle θ be small since the spatial separability (which will be described later) between the particulate components within the liquid sample S increases with decreasing contact angle θ. By enhancing the hydrophilicity and the wettability of the flat surface 4a so as to reduce the surface tension γSL between the flat surface 4a and the liquid sample S, the contact angle θ can be reduced to 10 degrees or smaller.

Next, in the drying step S2, the substrate 4 having the liquid droplet formed thereon is kept still without being moved, and the liquid droplet is dried. In order to shorten the drying time of the liquid droplet, the substrate 4 may be moderately heated, or the substrate 4 may be set still within a sealed space and moisture may be absorbed from within the sealed space.

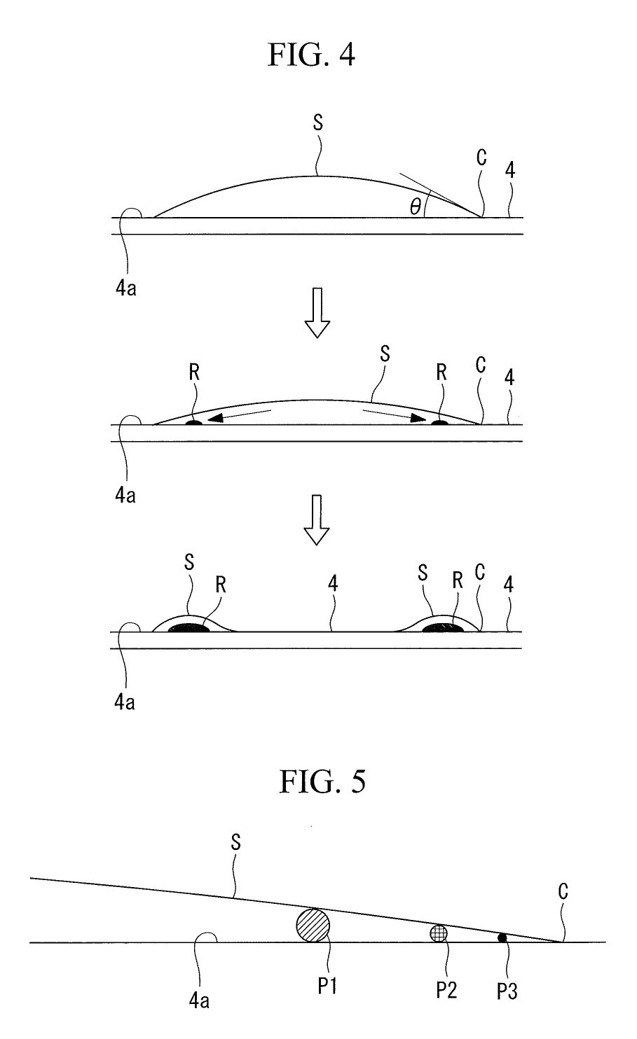

As shown in FIG. 4, in the liquid-droplet drying process, the liquid sample S evaporates while a contact line C remains fixed, so that the liquid droplet forms therein a radial liquid flow toward the contact line C in the radially outer direction from the center thereof. The contact line C is a circular outer edge of the liquid droplet at the interfacial boundary between the liquid droplet and the flat surface 4a. By means of the radial liquid flow in the liquid droplet, the solute and the particulate components contained in the liquid are transported toward the contact line C, and the transported particulate components become deposited near the contact line C. Consequently, ring-shaped deposits (referred to as “deposit rings” hereinafter) R substantially concentric with the contact line C are formed along the dried mark of the liquid droplet. This phenomenon is known as a coffee stain phenomenon and occurs as a result of a liquid flow produced from the central region of the liquid droplet toward the contact line C due to the evaporation rate of the liquid near the contact line C being greater than the evaporation rate of the liquid in the central region of the liquid droplet.

As mentioned above, the liquid sample S contains particulate components having various particle diameters, such as cells, bacteria, mycoplasmas, viruses, and proteins. As shown in FIG. 5, the transport positions, in the radial direction, of particulate components P1, P2, and P3 transported within the liquid droplet are dependent on the particle diameters of the particulate components P1, P2, and P3, and particulate components with smaller particle diameters are transported closer to the contact line C. Therefore, the particulate components P1, P2, and P3 having different particle diameters are deposited at different positions in the radial direction, thereby respectively forming deposit rings R1, R2, and R3 having different radii.

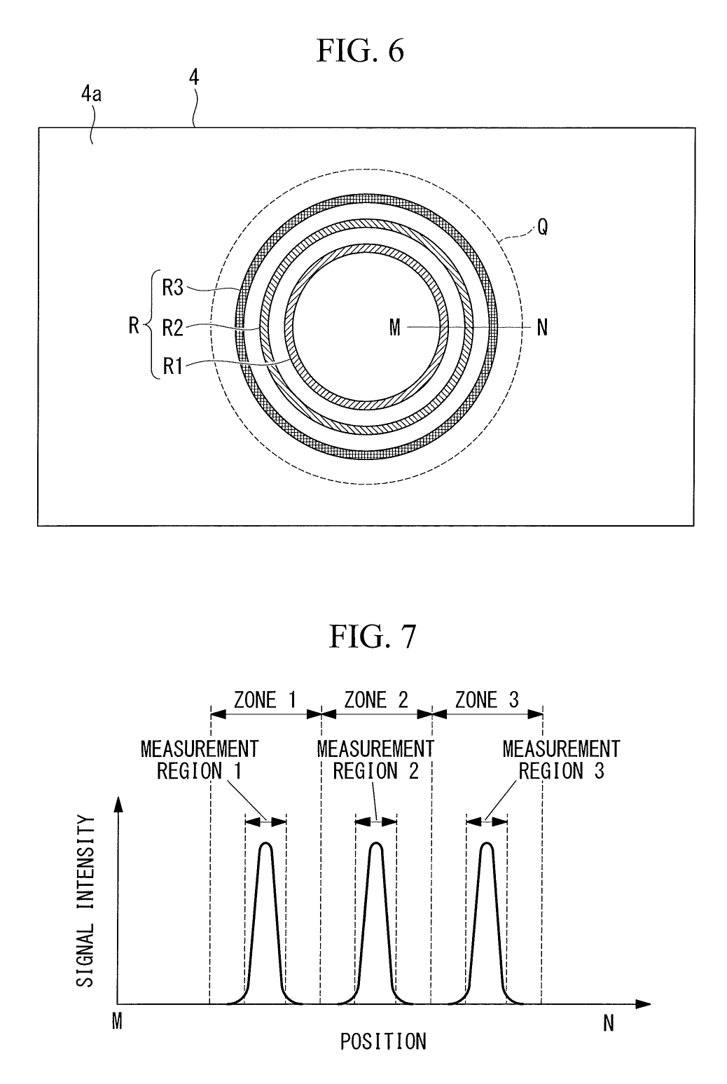

As a result, as shown in FIG. 6, the dried mark of the liquid droplet includes a plurality of concentrically-arranged deposit rings R1, R2, and R3 having different radii. For example, the deposit ring R1 formed of cells, the deposit ring R2 formed of bacteria, and the deposit ring R3 formed of viruses and proteins are formed in that order from the inner side. Accordingly, in the drying step S2, the particulate components contained in the liquid sample S can be spatially separated on the flat surface 4a in accordance with the particle diameters thereof.

More specifically, a particulate component Pa having a particle diameter Da and a particulate component Pb having a particle diameter Db (Db>Da) will be discussed. A deposit ring Ra formed from the particulate component Pa and a deposit ring Rb formed from the particulate component Pb have different radii ra and rb, respectively, and a distance d=ra−rb in the radial direction is produced between the deposit ring Ra and the deposit ring Rb. The distance d is dependent on the contact angle θ, and the relationship between the distance d and the contact angle θ can be formulated by being substantially expressed with the following expression (2):

(Db−Da)/d=tan(θ/2) (2)

Expression (2) indicates that, as the contact angle θ decreases, the distance d increases, and the spatial separability between the particulate components Pa and Pb becomes higher. Specifically, in a case where the contact angle θ is 10 degrees, the distance d between the deposit ring R1 formed of cells and the deposit ring R2 formed of bacteria is calculated to be about 100 μm to 200 μm, and the distance d between the deposit ring R2 formed of bacteria and the deposit ring R3 formed of proteins is calculated to be about 10 μm from expression (2) above. It assumed that the particle diameter of cells is between 10 μm and 20 μm, the particle diameter of bacteria is 1 μm, the particle diameter of viruses is 0.1 μm, and the particle diameter of proteins is smaller than 0.01 μm. Accordingly, as compared with the spatial resolution of the microscope 2, there are sufficiently large distances d among the deposit rings R1, R2, and R3 formed of particulate components of various types.

Next, in the preliminary measuring step S3, a vibrational spectrum is measured by the microscope 2 along a line segment MN set within the dried mark, and an intensity profile is created by the control device 3 from the acquired vibrational spectrum. The line segment MN is set in a straight line in the radial direction within the dried mark so as to traverse the plurality of deposit rings R1, R2, and R3 in the radial direction. The microscope 2 radiates probe light onto the dried mark while scanning it along the line segment MN, so as to measure the vibrational spectrum at each position on the line segment MN. The control device 3 plots the intensity of an arbitrary frequency range in the vibrational spectrum acquired by the microscope 2 in correspondence with the measurement position on the line segment MN, thereby creating an intensity profile indicating the intensity of the vibrational spectrum on the line segment MN, as shown in FIG. 7.

Because the intensity of the vibrational spectrum in the intensity profile corresponds to the density of each particulate component, the positions of the deposit rings R1, R2, and R3 can be determined by recognizing the spatial distribution of the deposit rings R1, R2, and R3 within the dripping range Q from the intensity profile. For example, the line segment MN can be set by using the microscope 2 to acquire a bright-field image of the dripping range Q, displaying the acquired image on a display unit, and allowing the user to input the line segment MN to the displayed image by using a graphical user interface (GUI).

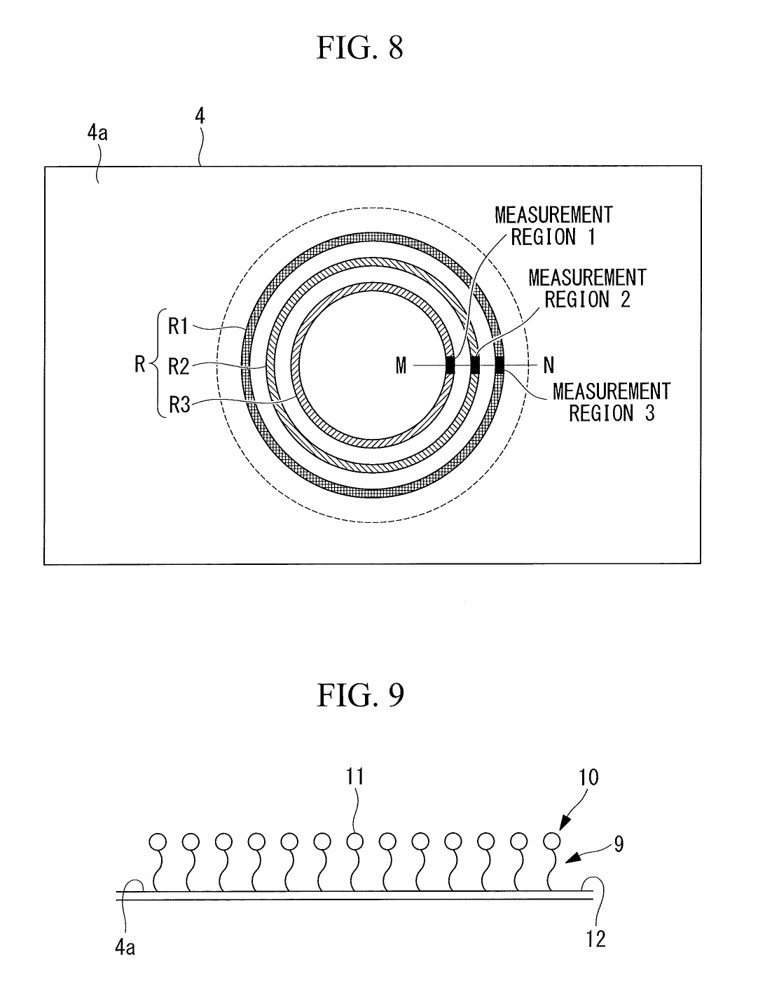

Next, in the measurement-region setting step S4, the control device 3 divides the line segment MN into a plurality of zones 1, 2, and 3 based on the intensity profile such that the deposit rings R1, R2, and R3 are respectively included in the zones 1, 2, and 3. For example, the positions where the intensity of the vibrational spectrum is at the minimum are set as the boundaries of the zones 1, 2, and 3. Then, measurement regions 1, 2, and 3 are set such that the positions where the intensity of the vibrational spectrum is at the maximum are preferably included within the zones 1, 2, and 3. The measurement regions 1, 2, and 3 may be set automatically by the control device 3 or may be set manually by the user. Furthermore, as shown in FIG. 8, the measurement regions 1, 2, and 3 may be two-dimensional regions having dimensions also in the direction intersecting the line segment MN.

Subsequently, in the measuring step S5, vibrational spectra are sequentially measured in the set measurement regions 1, 2, and 3 by using the microscope 2. Thus, the vibrational spectra of the various components contained in the liquid sample S can be individually measured in accordance with the respective particle diameters. For example, the vibrational spectrum of cells in the measurement region 1, the vibrational spectrum of bacteria in the measurement region 2, and the vibrational spectrum of proteins and viruses in the measurement region 3 can be individually measured. In this case, the vibrational spectra are measured at many measurement positions while scanning the probe light L in the measurement regions 1, 2, and 3, so that a large number of vibrational spectra with respect to the measurement regions 1, 2, and 3 are acquired.

Then, in the classifying step S6, the large number of vibrational spectra obtained from the measurement regions 1, 2, and 3 in the measuring step S5 are classified into a plurality of clusters in accordance with multivariate analysis. Examples of the multivariate analysis include a clustering method, such as the K-means method or the HCA (hierarchy cluster analysis) method, or the NMF (nonnegative matrix factorization) method. By using such multivariate analysis, the data of the large number of vibrational spectra acquired in the measuring step S5 is classified into a plurality of clusters such that vibrational spectra having similar waveforms belong to the same cluster.

Next, in the identifying step S7, the particulate components are identified by comparing the vibrational spectra classified into the respective clusters with the vibrational spectra of known components. Standard vibrational spectra of particulate components, such as cells, bacteria, viruses, and proteins, contained in the liquid sample S are acquired in advance and are stored in the control device 3 as a database. In the identifying step S7, the particulate components are identified by searching for, within the database, vibrational spectra with waveforms identical or similar to those of the vibrational spectra classified into the respective clusters.

With the liquid-sample component analysis method according to this embodiment, the particulate components contained in the liquid sample S are spatially separated in accordance with the respective particle diameters thereof by simply waiting for the liquid droplet to dry after the liquid sample S is dripped onto the flat surface 4a. Therefore, when analyzing a mixed sample containing components that have various particle diameters and that are chemically different from one another, such as cells, bacteria, viruses, and proteins, a complicated vibrational spectrum of the mixed sample to be observed can be simplified, so that high analysis performance can be achieved. Accordingly, a biochemical separation and refinement operation, such as centrifugal separation and chromatography, performed as pretreatment for liquid analysis in the related art is not necessary, which is advantageous in that relatively simple component analysis can be performed by using a compact low-cost device.

Furthermore, since the liquid droplet has a small volume (several μL to several hundreds of μL), the time required for drying the liquid droplet is about several minutes. This is advantageous in that the particulate components can be spatially separated in an extremely short period of time and that the component analysis can be performed quickly. Moreover, by utilizing the coffee ring phenomenon, the particulate components in the liquid sample S gather in small volumes of the deposit rings R1, R2, and R3, and the particulate components are concentrated at the deposit rings R1, R2, and R3. This is advantageous in that even a particulate component contained at only a small amount in the liquid sample S can be analyzed with high sensitivity.

As an alternative to this embodiment in which the vibrational spectrum is preliminarily measured at the dried mark of the liquid droplet and the measurement regions are set based on the acquired intensity profile, a standard map indicating the spatial distribution of a deposit ring, which is formed of a standard component having a known particle diameter, in the dripping range Q may be preliminarily created (standard-map creating step) prior to performing steps S1 to S7, and the measurement regions 1, 2, and 3 may be set based on the standard map.

Specifically, the standard map is created by preparing a standard solution by dispersing a plurality of known types of particles having particle diameters on the order of subnanometers to micrometers in a liquid, such as water, performing the dripping step S1 and the drying step S2 by using the prepared standard solution, and measuring the distribution of the deposit rings R1, R2, and R3 formed on the flat surface 4a. Consequently, it is possible to estimate where in the dripping range Q a particulate component to be analyzed will form a deposit ring, so that a vibrational spectrum of the deposit ring that includes the particulate component to be analyzed can be selectively measured.

Furthermore, in a case where the dripping range Q is small in this embodiment, probe light may be simultaneously and uniformly radiated onto the entire dripping range Q so as to acquire a two-dimensional intensity profile of the entire dripping range Q. For example, the so-called ATR measurement method may be performed by causing probe light to enter a prism serving as the substrate 4, causing the probe light to be totally reflected by the flat surface 4a so as to generate evanescent light near the flat surface 4a, and measuring a Raman scattering spectrum or an infrared absorption spectrum generated by the evanescent light. Alternatively, the two-dimensional intensity profile may be acquired by two-dimensionally scanning probe light within the dripping range Q and converting the intensities of vibrational spectra obtained at respective positions into a two-dimensional image as pixel values.

Next, a modification of this embodiment will be described.

### (First Modification)

As shown in FIG. 9, in a first modification of this embodiment, the substrate 4 used has a molecular layer 9 and a metallic particle layer 10 formed on the flat surface 4a in that order from the flat surface 4a side. The molecular layer 9 is, for example, a self-organizing monomolecular film, such as alkanethiol molecules, formed on the flat surface 4a and preferably has a small film thickness of about several nanometers. The molecules constituting the molecular layer 9 each have a hydrophilic group, such as a hydroxyl group or an amino group, at the opposite end from the flat surface 4a. The metallic particle layer 10 is composed of metal, such as silver or copper, and is constituted of metallic particles 11 existing as single particles or composite particles. The particle diameter of each metallic particle 11 is preferably between 10 nm and several tens of nanometers. The metallic particles 11 are fixed on the flat surface 4a by being bonded to the ends of the molecular layer 9.

The metallic particles 11 generate an enhanced electric field in the vicinity thereof and significantly enhance Raman scattering or infrared absorption of particulate components in contact with or in the vicinity of the metallic particles 11. Therefore, the vibrational spectrum from each particulate component can be measured within a short period of time and with high sensitivity.

In this modification, a metallic layer 12 that covers the flat surface 4a may be formed on the flat surface 4a, and the molecular layer (spacer layer) 9 and the metallic particle layer 10 may be formed on the metallic layer 12 in that order from the flat surface 4a side. Accordingly, the gap between the metallic layer 12 and the metallic particles 11 and the electric field in the vicinity thereof can be further enhanced, thereby further enhancing the vibrational spectrum.

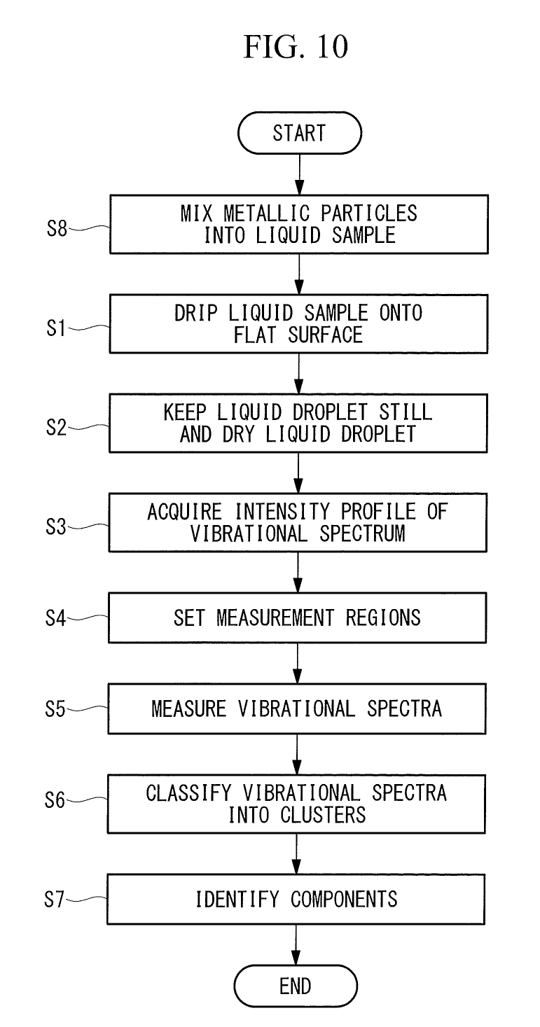

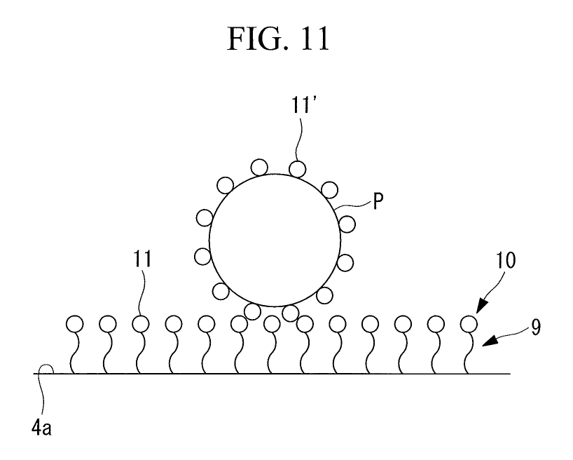

As shown in FIG. 10, in this embodiment, a metallic-particle mixing step S8 of mixing nanometer-size metallic particles into the liquid sample S may be performed prior to the dripping step S1, and the liquid sample S containing the metallic particles may be used in the dripping step S1. In the metallic-particle mixing step S8, metallic particles similar to the metallic particles 11 used in the aforementioned metallic particle layer 10 can be used. The metallic particles nonspecifically adsorb to the particulate components, such as bacteria, viruses, and proteins, in the liquid sample S. As shown in FIG. 11, by using such a liquid sample S, a particulate component P that has adsorbed metallic particles 11′ comes into contact with or close to the metallic particle layer 10 on the flat surface 4a so that an enhanced electric field is generated, thereby further enhancing the vibrational spectrum. In a case where a liquid sample S mixed with the metallic particles 11′ is to be used, a substrate 4 not having the metallic particle layer 10 or the metallic layer 12 may be used.

### (Second Modification)

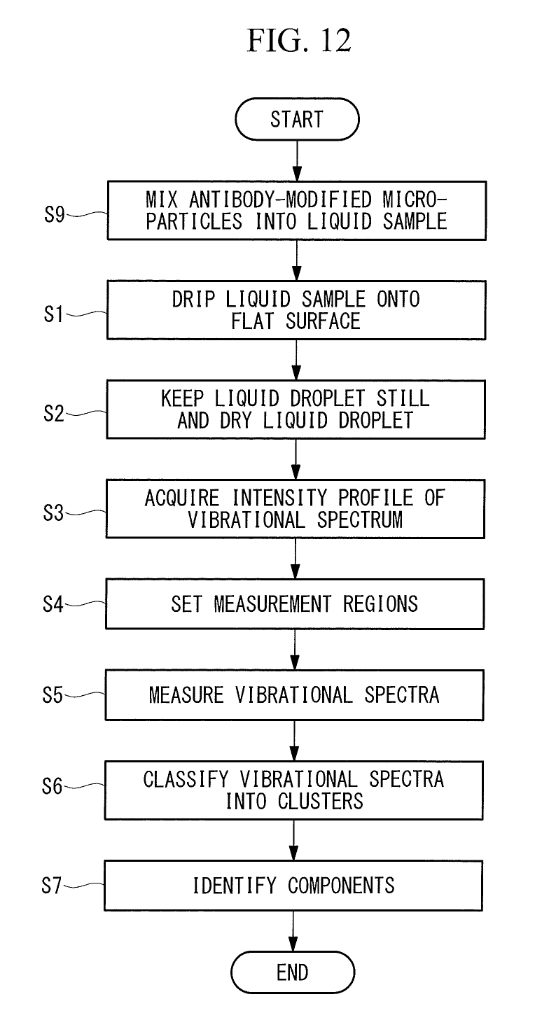

As shown in FIG. 12, in a second modification of this embodiment, an antibody-modified-particle mixing step S9 of mixing the liquid sample S with micro-particles (antibody modified particles) I, II, and III, each having a micrometer-size particle diameter and surface-modified with an antibody, is performed prior to the dripping step S1, and the liquid sample S containing the micro-particles I, II, and III is used in the dripping step S1.

The micro-particles I, II, and III are, for example, spherical particles composed of polystyrene, polymethyl methacrylate (PMMA), or silica. The particle diameter of each micro-particle is preferably about several micrometers to 100 μm. An antibody that specifically bonds with a particulate component to be analyzed in the liquid sample S is fixed to the surface of each of the micro-particles I, II, and III, and the particulate component to be analyzed is fixed to the surface of the corresponding one of the micro-particles I, II, and III in the liquid sample S via the antibody.

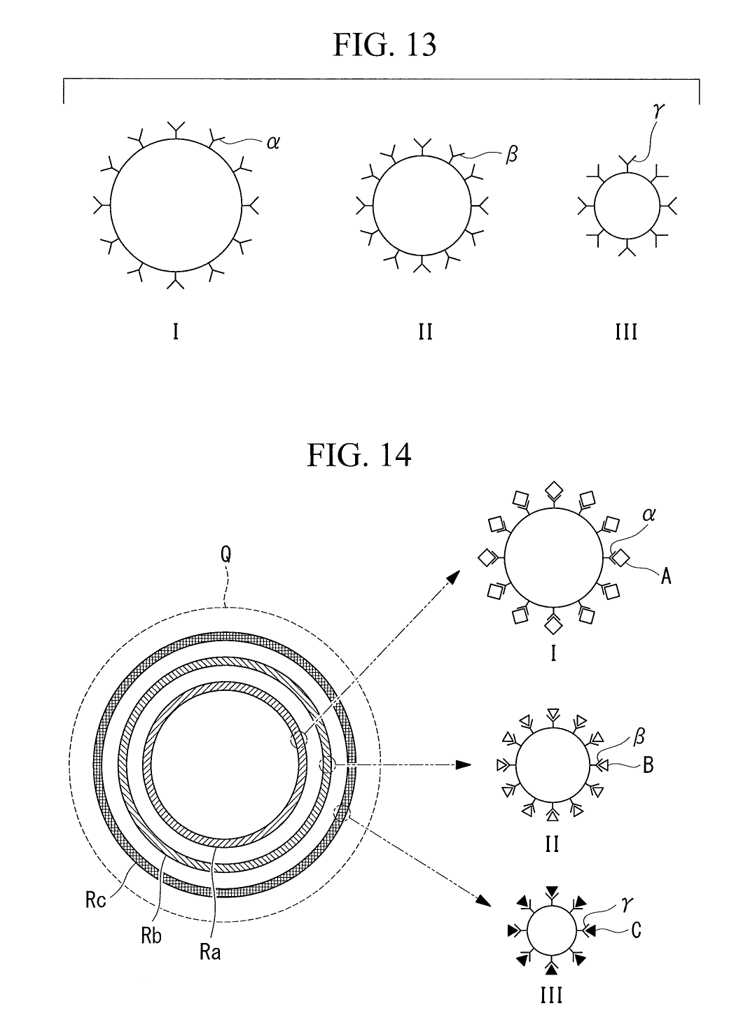

As shown in FIG. 13, three types of micro-particles I, II, and III having particle diameters different from one another are simultaneously mixed in the liquid sample S. The micro-particles I, II, and III have antibodies α, β, and γ fixed thereto, which are targeted at different types of proteins from particle diameter to particle diameter. For example, the micro-particle I with a particle diameter Di has fixed thereto the antibody a against a protein A, the micro-particle II with a particle diameter Dii has fixed thereto the antibody p against a protein B, and the micro-particle III with a particle diameter Diii has fixed thereto the antibody γ against a protein C. The magnitude relationship among the diameters Di, Dii, and Diii is as follows: Diii<Dii<Di.

As shown in FIG. 14, in the dried mark of the liquid droplet of such a liquid sample S, a deposit ring Ra formed from the micro-particle I bonded with the protein A, a deposit ring Rb formed from the micro-particle II bonded with the protein B, and a deposit ring Rc formed from the micro-particle III bonded with the protein C are arranged in that order from the center toward the contact line C in the radial direction.

Because the particle diameters of proteins range between several nanometers and several tens of nanometers, it is difficult to spatially separate different types of proteins from each other by only utilizing the differences in particle diameters of the proteins in accordance with this method. Thus, the micro-particles having larger particle diameters than the proteins are used, and the proteins A, B, and C of different types are bonded with the micro-particles I, II, and III having different particle diameters, so that the proteins having substantially identical particle diameters can be clearly spatially separated from one another. Consequently, the vibrational spectra of the three types of proteins A, B, and C can be differentiated from each other and can be measured with high accuracy without having to label them with different labeling materials for the respective antibodies α, β, and γ.

Furthermore, a vibrational spectrum derived from the protein A can be obtained by selecting a material, as the micro-particle I, such that the vibrational spectrum of the particle I does not interfere with the observation of the vibrational spectrum of the target protein or by preliminarily acquiring the vibrational spectrum of the micro-particle I alone and detecting a difference between the vibrational spectrum acquired from the deposit ring Ra and the vibrational spectrum of the micro-particle I alone. Likewise, a vibrational spectrum derived from the protein B can be obtained by using the vibrational spectrum of the micro-particle II alone, and a vibrational spectrum derived from the protein C can be obtained by using the vibrational spectrum of the micro-particle III alone.

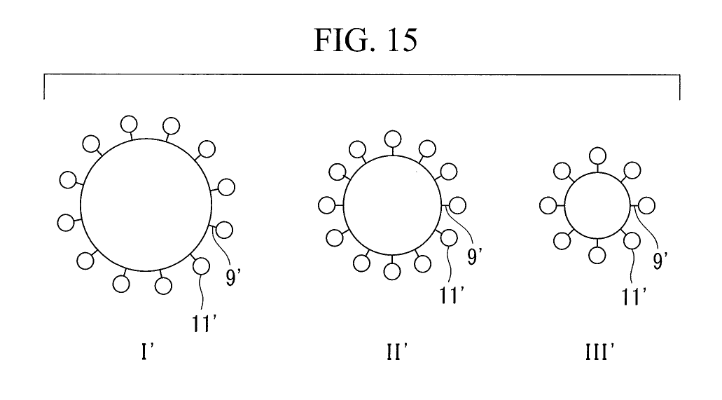

In this modification, the antibody-modified-particle mixing step S9 may involve mixing the liquid sample S with micro-particles (metal-modified particles) I′, II′, and III′, whose surfaces have nanometer-size metallic particles 11′ fixed thereto, together with the micro-particles I, II, and III, as shown in FIG. 15. The micro-particle I′ has substantially the same particle diameter as the micro-particle I, the micro-particle II′ has substantially the same particle diameter as the micro-particle II, and the micro-particle III′ has substantially the same particle diameter as the micro-particle III. The metallic particles 11′ are fixed to the micro-particles I′, II′, and III′ via molecular films 9′ that cover the surfaces of the micro-particles I′, II′, and III′. The molecular films 9′ are, for example, self-organizing monomolecular films and are similar to the molecular layer 9 described above.

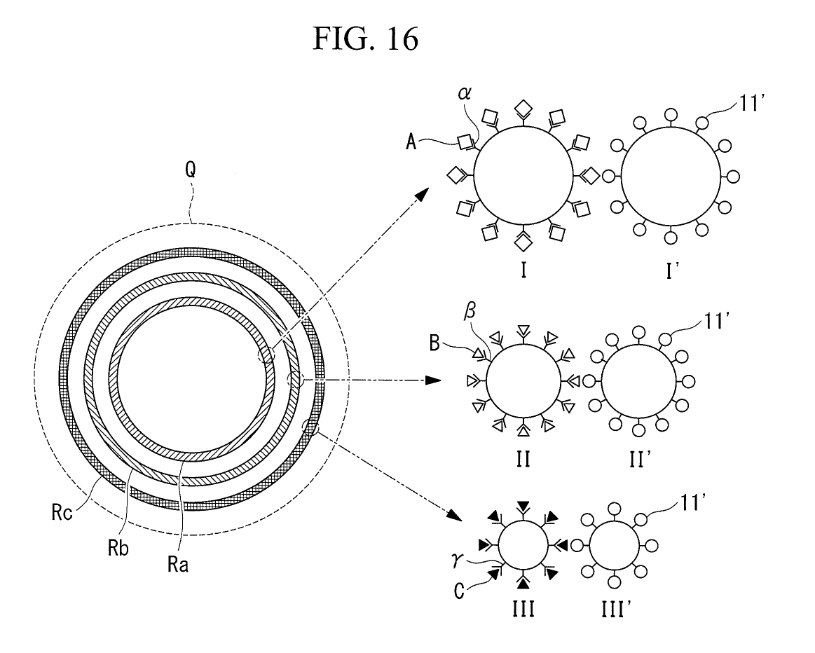

As shown in FIG. 16, in the dried mark of the liquid droplet of the liquid sample S mixed with the micro-particles I, II, and III and the micro-particles I′, II′, and III′ in this manner, the micro-particle I and the micro-particle I′ form the same deposit ring Ra, the micro-particle II and the micro-particle II′ form the same deposit ring Rb, and the micro-particle III and the micro-particle III′ form the same deposit ring Rc. Accordingly, the metallic particles 11′ exist near the particulate components to be analyzed in the respective deposit rings Ra, Rb, and Rc, so that the vibrational spectra can be enhanced, thereby further enhancing the analytical sensitivity.

As a result, the above-described embodiment leads to the following aspect.

An aspect of the present invention provides a liquid-sample component analysis method including a dripping step of dripping a liquid sample onto a flat horizontal surface; a drying step of drying a liquid droplet of the liquid sample formed on the horizontal surface as a result of the dripping step while keeping the liquid droplet still so as to obtain a plurality of concentrically-arranged ring-shaped deposits formed on the horizontal surface and composed of components having different particle diameters; and a measuring step of measuring a vibrational spectrum in each region including only one of the deposits so as to individually acquire vibrational spectra of the plurality of deposits.

According to this aspect, when the liquid droplet of the liquid sample formed on the horizontal surface in the dripping step is dried in the drying step, the components in the liquid droplet become deposited along the outer edge of the liquid droplet due to a coffee stain phenomenon, so that ring-shaped deposits are obtained in the dried mark of the liquid droplet. The deposited positions of the components in the dried mark vary depending on the particle diameters thereof. A large particulate component is deposited toward the center, whereas a small particulate component is deposited at the outer side in the radial direction. Therefore, the various particulate components in the liquid droplet are spatially separated on a two-dimensional surface in accordance with the particle diameters thereof as a result of the drying step.

In the subsequent measuring step, the vibrational spectra of the plurality of concentrically-arranged deposits are individually measured for the respective deposits so that the vibrational spectra of specific particulate components can be measured.

Accordingly, the various particulate components in the liquid droplet are spatially separated by simply forming and drying the liquid droplet, so that the spectral shape of the vibrational spectrum of the mixed sample to be observed is simplified and the particulate components are concentrated, whereby small amounts of particulate components can be analyzed with high sensitivity. Moreover, since a small amount of liquid droplet dries within a short period of time, rapid analysis can be performed.

In the above aspect, the liquid-sample component analysis method may further include a preliminary measuring step of measuring a vibrational spectrum along a line segment traversing the plurality of deposits in a radial direction so as to obtain an intensity profile in which a position on the line segment and the intensity of the vibrational spectrum acquired at the position are associated with each other; and a measurement-region setting step of dividing the line segment into a plurality of zones, which respectively include the deposits, in the radial direction based on the intensity profile obtained in the preliminary measuring step and setting measurement regions for the respective zones. The setting step may include measuring vibrational spectra for the respective measurement regions set in the measurement-region setting step.

Accordingly, because a vibrational spectrum intensifies at a position where a deposit is present on the line segment, the positions of the plurality of deposits within the dried mark can be identified based on the intensities of vibrational spectra in the intensity profile. Then, zones are set such that only one deposit is included in one zone, and measurement regions are set for the respective deposits by setting the measurement regions for the respective zones, whereby vibrational spectra for component analysis can be measured.

In the above aspect, the liquid-sample component analysis method may further include a standard-map creating step of forming the deposits on the horizontal surface by using a standard solution containing a standard component having a known particle diameter and creating a standard map indicating the distribution of the obtained deposits, which are composed of the standard component, on the horizontal surface. The measuring step may include setting regions where the vibrational spectra are measured based on the standard map.

Accordingly, by using the standard map, the position where a particulate component to be analyzed forms a deposit is identified from the magnitude relationship between the particle diameter of the particulate component to be analyzed and the particle diameter of the standard component, whereby the vibrational spectrum of the particulate component to be analyzed can be efficiently measured.

In the above aspect, the liquid-sample component analysis method may further include a classifying step of classifying the vibrational spectra acquired in the measuring step into a plurality of clusters in accordance with multivariate analysis; and an identifying step of identifying a component contained in the liquid sample based on the vibrational spectra classified into the respective clusters in the classifying step and a preliminarily-acquired vibrational spectrum of a known component.

Accordingly, by using multivariate analysis, the large number of vibrational spectra obtained from the deposits are classified such that similar vibrational spectra belong to the same cluster. Then, by comparing the vibrational spectra belonging to each cluster with the vibrational spectrum of the known component, the particulate component constituting each deposit can be identified.

In the above aspect, a metallic layer covering the horizontal surface may be formed on the horizontal surface, a metallic particle layer formed of metallic particles having a nanometer-size particle diameter may be formed on the metallic layer, and a spacer layer that fixes the metallic particles to the metallic layer may be interposed between the metallic layer and the metallic particle layer.

Accordingly, the particulate components come close to the metallic particles so that the intensities of the vibrational spectra from the particulate components are enhanced, thereby further enhancing the analytical sensitivity. Moreover, with the metallic film formed near the metallic particles, the vibrational spectra can be further enhanced.

In the above aspect, the liquid-sample component analysis method may further include a metallic-particle mixing step of mixing the liquid sample with metallic particles each having a nanometer-size particle diameter, the metallic-particle mixing step being performed prior to the dripping step.

Accordingly, the metallic particles mixed in the liquid sample nonspecifically bond with a particulate component in the liquid sample. Thus, the vibrational spectrum from the particulate component is effectively enhanced by the metallic particles bonded with the particulate component, thereby further enhancing the analytical sensitivity.

In the above aspect, the liquid-sample component analysis method may further include an antibody-modified-particle mixing step of mixing the liquid sample with antibody-modified particles each having a micrometer-size particle diameter and having an antibody fixed on a surface thereof, the antibody-modified-particle mixing step being performed prior to the dripping step.

Accordingly, a specific particulate component is fixed to the surface of each micrometer-size antibody-modified particle via the antibody, and is separated in the drying step based on the particle diameter of the antibody-modified particle. Thus, the spatial separability between the specific particulate component and another particulate component, specifically, another particulate component having an identical particle diameter, can be enhanced.

In the above aspect, the antibody-modified-particle mixing step may include mixing the liquid sample with a plurality of types of the antibody-modified particles having different particle diameters and having antibodies, which are targeted at different particulate components, fixed to surfaces thereof.

Accordingly, the spatial separability between particulate components having the same particle diameter can be enhanced based on a difference in the particle diameters of the antibody-modified particles.

In the above aspect, the antibody-modified-particle mixing step may include mixing the liquid sample with metal-modified particles that have metallic particles with a nanometer-size particle diameter fixed on surfaces thereof and that have a particle diameter substantially identical to that of the antibody-modified particles.

Accordingly, since the antibody-modified particles and the metal-modified particles having a specific particulate component fixed thereto coexist close to each other within the same deposit, the vibrational spectrum from the specific particulate component can be enhanced by the metallic particles, thereby further enhancing the analytical sensitivity.

The present invention is advantageous in that it enables simple, rapid, highly-sensitive component analysis of a liquid sample containing various types of components.

### REFERENCE SIGNS LIST

4a flat surface (horizontal surface)
9 molecular layer (spacer layer)
10 metallic particle layer
11 metallic particle
12 metallic layer
S liquid sample
R, R1, R2, R3, Ra, Rb, Rc deposit ring (deposit)
I, II, III micro-particle (antibody-modified particle)
I′, II′, III′ micro-particle (metal-modified particle)
S1 dripping step
S2 drying step
S3 preliminary measuring step
S4 measurement-region setting step
S5 measuring step
S6 classifying step
S7 identifying step
S8 metallic-particle mixing step
S9 antibody-modified-particle mixing step

1. A liquid-sample component analysis method comprising:
a dripping step of dripping a liquid sample onto a flat horizontal surface;
a drying step of drying a liquid droplet of the liquid sample formed on the horizontal surface as a result of the dripping step while keeping the liquid droplet still so as to obtain a plurality of concentrically-arranged ring-shaped deposits formed on the horizontal surface and composed of components having different particle diameters; and
a measuring step of measuring a vibrational spectrum in each region including only one of the deposits so as to individually acquire vibrational spectra of the plurality of deposits.

2. The liquid-sample component analysis method according to claim 1, further comprising:
a preliminary measuring step of measuring a vibrational spectrum along a line segment traversing the plurality of deposits in a radial direction so as to obtain an intensity profile in which a position on the line segment and the intensity of the vibrational spectrum acquired at the position are associated with each other; and
a measurement-region setting step of dividing the line segment into a plurality of zones, which respectively include the deposits, in the radial direction based on the intensity profile obtained in the preliminary measuring step and setting measurement regions for the respective zones,
wherein the setting step includes measuring vibrational spectra for the respective measurement regions set in the measurement-region setting step.

3. The liquid-sample component analysis method according to claim 1, further comprising:
a standard-map creating step of forming the deposits on the horizontal surface by using a standard solution containing a standard component having a known particle diameter and creating a standard map indicating the distribution of the obtained deposits, which are composed of the standard component, on the horizontal surface,
wherein the measuring step includes setting regions where the vibrational spectra are measured based on the standard map.

4. The liquid-sample component analysis method according to claim 1, further comprising:
a classifying step of classifying the vibrational spectra acquired in the measuring step into a plurality of clusters in accordance with multivariate analysis; and
an identifying step of identifying a component contained in the liquid sample based on the vibrational spectra classified into the respective clusters in the classifying step and a preliminarily-acquired vibrational spectrum of a known component.

5. The liquid-sample component analysis method according to claim 1,
wherein a metallic layer covering the horizontal surface is formed on the horizontal surface, a metallic particle layer formed of metallic particles having a nanometer-size particle diameter is formed on the metallic layer, and a spacer layer that fixes the metallic particles to the metallic layer is interposed between the metallic layer and the metallic particle layer.

6. The liquid-sample component analysis method according to claim 1, further comprising:
a metallic-particle mixing step of mixing the liquid sample with metallic particles each having a nanometer-size particle diameter, the metallic-particle mixing step being performed prior to the dripping step.

7. The liquid-sample component analysis method according to claim 1, further comprising:
an antibody-modified-particle mixing step of mixing the liquid sample with antibody-modified particles each having a micrometer-size particle diameter and having an antibody fixed on a surface thereof, the antibody-modified-particle mixing step being performed prior to the dripping step.

8. The liquid-sample component analysis method according to claim 7,
wherein the antibody-modified-particle mixing step includes mixing the liquid sample with a plurality of types of the antibody-modified particles having different particle diameters and having antibodies, which are targeted at different particulate components, fixed to surfaces thereof.

9. The liquid-sample component analysis method according to claim 7,
wherein the antibody-modified-particle mixing step includes mixing the liquid sample with metal-modified particles that have metallic particles with a nanometer-size particle diameter fixed on surfaces thereof and that have a particle diameter substantially identical to that of the antibody-modified particles.
